(19) United States
(12) Patent Application Publication
(10) Pub. No.: US 2023/0329884 A1
(43) Pub. Date: Oct. 19, 2023

(54) PROSTHETIC KNEE JOINT

(71) Applicant: TEH LIN PROSTHETIC & ORTHOPAEDIC INC., New Taipei City (TW)

(72) Inventor: Jian-Yu CHEN, New Taipei City (TW)

(21) Appl. No.: 17/943,069

(22) Filed: Sep. 12, 2022

(30) Foreign Application Priority Data
Apr. 15, 2022 (TW) 111114521

(51) Int. Cl. A61F 2/64

(52) U.S. Cl. CPC A61F 2/644; A61F 2002/5072

(57) ABSTRACT

A prosthetic knee joint includes a lower leg connecting part, a thigh connecting part, a linkage assembly and a biasing unit. The lower leg connecting part has first anterior and posterior link portions. The first posterior link portion has a slidable pivot shaft. The thigh connecting part has second anterior and second posterior link portions. The linkage assembly includes an anterior link pivotably connected between the first and second anterior link portions, and a posterior link pivotably connected between the first and second posterior link portions. The posterior link has a pivot linkage elongated to have upper and lower slot end sections. The slidable pivot shaft is slidably and rotatably engaged in the pivot linkage. The biasing unit abuts against the posterior link to bias upwardly the posterior link so as to urge the lower slot end section to engage with the slidable pivot shaft.

### CROSS-REFERENCE TO RELATED APPLICATION

This application claims priority of Taiwanese Patent Application No. 111114521, filed on Apr. 15, 2022.

### FIELD

The disclosure relates to a prosthetic knee joint, and more particularly to a prosthetic knee joint with a biasing unit providing a damping effect during walking.

### BACKGROUND

A conventional prosthetic knee joint includes a lower leg connecting part and a thigh connecting part which is coupled to the lower leg connecting part through a linkage assembly with an anterior link and a posterior link connected between the lower leg connecting part and the thigh connecting part. The lower leg connecting part, the thigh connecting part, the anterior link and the posterior link are cooperatively formed as a four-bar linkage mechanism. The thigh connecting part is movable relative to the lower leg connecting part between a stance position and a knee bending position. However, the conventional prosthetic knee joint with such four-bar linkage mechanism provides insufficient damping against an impact generated as a result of contact with the ground during walking.

### SUMMARY

Therefore, an object of the disclosure is to provide a prosthetic knee joint that can alleviate at least one of the drawbacks of the prior art.

According to the disclosure, the prosthetic knee joint includes a lower leg connecting part, a thigh connecting part, a linkage assembly and a biasing unit. The lower leg connecting part has a first anterior link portion and a first posterior link portion. The first posterior link portion has a slidable pivot shaft which extends in a left-right direction. The thigh connecting part is disposed above the lower leg connecting part and has a second anterior link portion and a second posterior link portion. The linkage assembly is disposed between the lower leg connecting part and the thigh connecting part. The linkage assembly includes at least one anterior link which is pivotably connected between the first anterior link portion and the second anterior link portion, and a posterior link which is pivotably connected between the first posterior link portion and the second posterior link portion. The posterior link has a slotted pivot linkage which extends therethrough in the left-right direction for pivotally connecting and engaging with the slidable pivot shaft. The pivot linkage is elongated in an up-down direction to have an upper slot end section and a lower slot end section. The slidable pivot shaft is slidably and rotatably engaged in the pivot linkage such that, through pivoting of the linkage assembly, the thigh connecting part is movable relative to the lower leg connecting part between a stance position and a knee bending position, and such that the slidable pivot shaft is moved from the lower slot end section to the upper slot end section with an impact transmitted from the lower leg connecting part to cause a downward movement of the posterior link relative to the lower leg connecting part. The biasing unit abuts against the posterior link to urge movement of the thigh connecting part toward the stance position, and to bias upwardly the posterior link so as to urge the lower slot end section of the pivot linkage to engage with the slidable pivot shaft.

With the slidable engagement of the slidable pivot shaft with the pivot linkage, and the biasing unit biasing the posterior link upwardly and urging the pivot linkage to bring the lower slot end section to engage with the slidable pivot shaft, a damping effect is provided to reduce the impact when contacting with the ground. Also, an excess inflexion of the knee joint in the foot ground-contact phase is prevented for increased safety.

### BRIEF DESCRIPTION OF THE DRAWINGS

Other features and advantages of the disclosure will become apparent in the following detailed description of the embodiment with reference to the accompanying drawings, of which:

FIG. 1 is a perspective view illustrating an embodiment of a prosthetic knee joint according to the disclosure;

FIG. 2 is a perspective view of the embodiment viewed from another angle;

FIG. 3 is a schematic side view of the embodiment in a state when a thigh connecting part is in a stance position;

FIG. 4 is a partially-sectional side view of the embodiment in a state when the thigh connecting part is in the stance position;

FIG. 5 is a schematic side view of the embodiment in a state when the thigh connecting part is in a knee bending position;

FIG. 6 is a partially-sectional side view of the embodiment in a state when the thigh connecting part is in the knee bending position;

FIG. 7 is a schematic side view of the embodiment in a state when a posterior link is moved downward relative to a lower leg connecting part to permit an upper slot end section of a pivot linkage to engage a slidable pivot shaft; and

FIG. 8 is a partially-sectional side view of the embodiment in a state as shown in FIG. 7.

### DETAILED DESCRIPTION

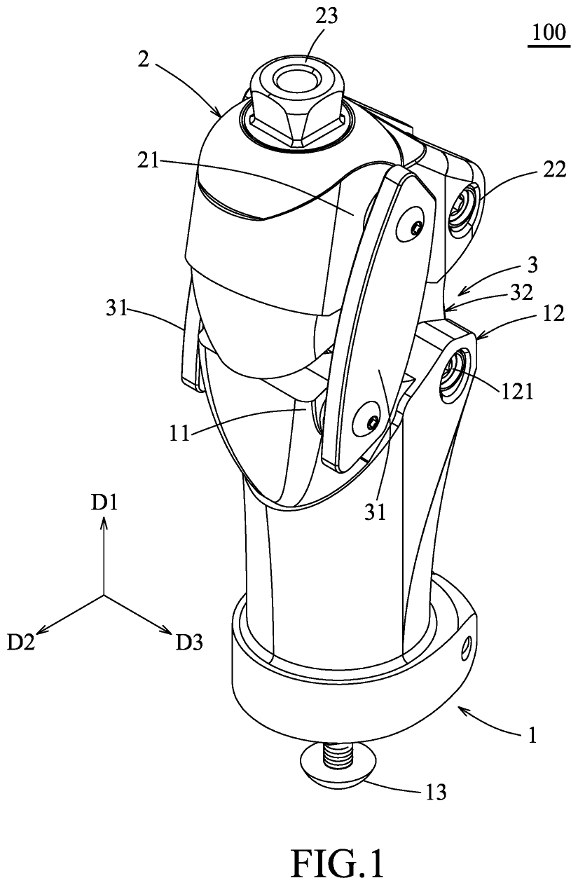

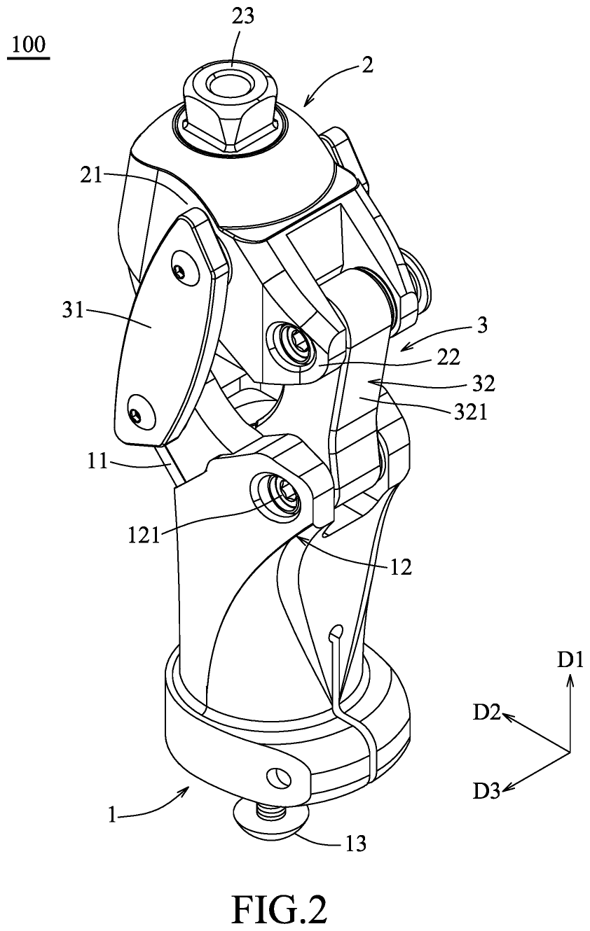

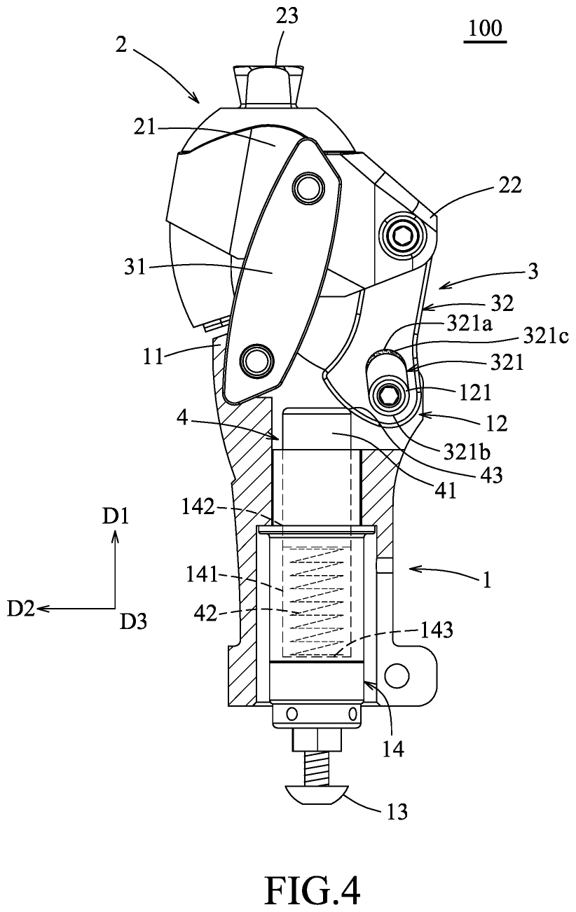

Referring to FIGS. 1 to 4, an embodiment of a prosthetic knee joint 100 includes a lower leg connecting part 1, a thigh connecting part 2, a linkage assembly 3 and a biasing unit 4.

The lower leg connecting part 1 substantially extends in an up-down direction (D1), and has a first anterior link portion 11 and a first posterior link portion 12 which are at an upper end thereof and opposite to each other in a front-rear direction (D2), and a lower leg connecting portion 13 which is at a lower end thereof for connecting a shin tube (not shown). The first posterior link portion 12 has a slidable pivot shaft 121 which extends in a left-right direction (D3).

The thigh connecting part 2 is disposed above the lower leg connecting part 1, and has a second anterior link portion 21 and a second posterior link portion 22 which are opposite to each other in the front-rear direction (D2), and a thigh connecting portion 23 which is at an upper end thereof for connecting a thigh socket (not shown).

The linkage assembly 3 is disposed between the lower leg connecting part 1 and the thigh connecting part 2. The linkage assembly 3 includes two anterior links 31 each of which is pivotably connected between the first anterior link portion 11 and the second anterior link portion 21, and a posterior link 32 which is pivotably connected between the first posterior link portion 12 and the second posterior link portion 22. The number of the anterior links 31 may be one or more than two. The posterior link 32 has a slotted pivot linkage 321 which extends therethrough in the left-right direction (D3) for pivotally connecting and engaging with the slidable pivot shaft 121. The pivot linkage 321 is elongated in the up-down direction (D1) to have an upper slot end section (321a) and a lower slot end section (321b). The slidable pivot shaft 121 is slidably and rotatably engaged in the pivot linkage 321. The posterior link 32 further has a cushion member (321c) disposed in the upper slot end section (321a) for the slidable pivot shaft 121 to abut thereagainst during the sliding movement of the slidable pivot shaft 121 along the pivot linkage 321. For example, the cushion member (321c) is made of polymeric material, such as foam material. Thus, when the posterior link 32 is moved downward to bring the pivot linkage 321 to move downward relative to the slidable pivot shaft 121 so as to cause the engagement of the slidable pivot shaft 121 with the upper slot end section (321a), the cushion member (321c) can provide a damping effect to the slidable pivot shaft 121. That is, the cushion member (321c) provides a damping effect when the slidable pivot shaft 121 slides along the pivot linkage 321 from the lower slot end section (321b) to the upper slot end section (321a).

Specifically, the slidable pivot shaft 121 is moved from the lower slot end section (321b) to the upper slot end section (321a) by a distance ranging from 5 mm to 7 mm. In this embodiment, the distance is 6 mm.

The biasing unit 4 abuts against the posterior link 32 to urge movement of the thigh connecting part 2 toward the stance position. The biasing unit 4 is attached to and abuts against a lower portion of the posterior link 32 at a torque application point 43 which is disposed anterior of the slidable pivot shaft 121. Specifically, the lower leg connecting part 1 has a central accommodation barrel 14 which has an upper opened end 142 and which extends in the up-down direction (D1) from the upper opened end 142 to terminate at a bottom barrel wall 143. The central accommodation barrel 14 defines an accommodation space 141 therein. The biasing unit 4 includes an abutting member 41 which is slidably received in the accommodation space 141 and which projects partially from the upper opened end 142 of the central accommodation barrel 14 to abut against the posterior link 32, and a biasing member 42 which is disposed in the accommodation space 141 and interposed between the bottom barrel wall 143 and the abutting member 41. The abutting member 41 has the torque application point 43 abutting against the lower portion of the posterior link 32. The biasing member 42 may be a compression spring.

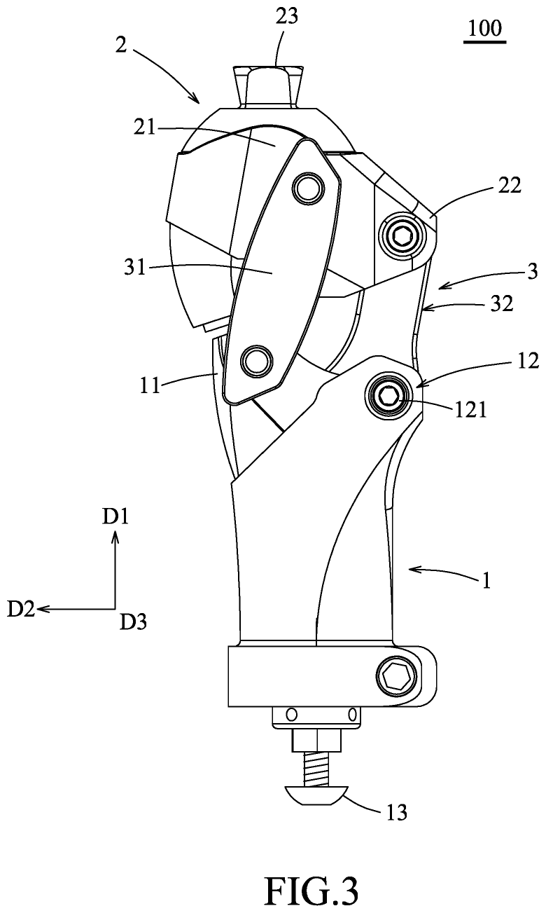

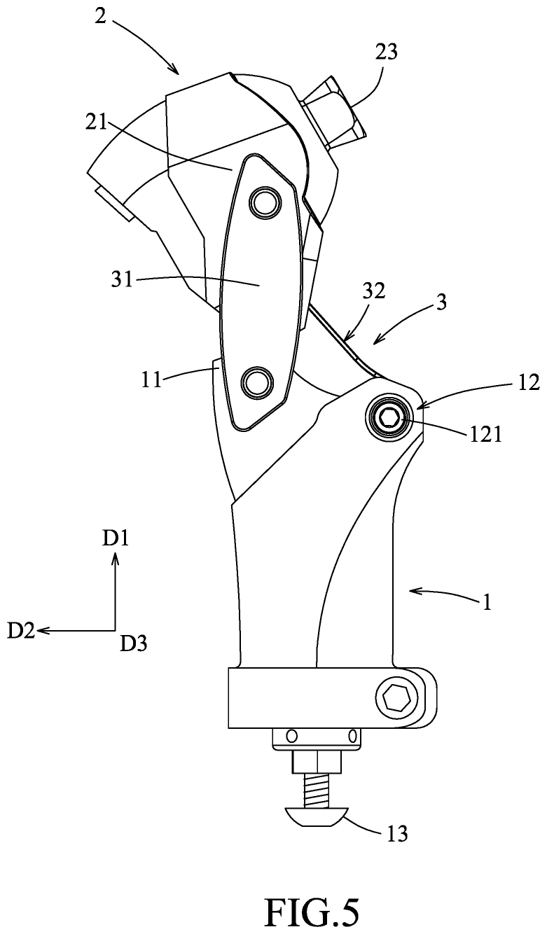

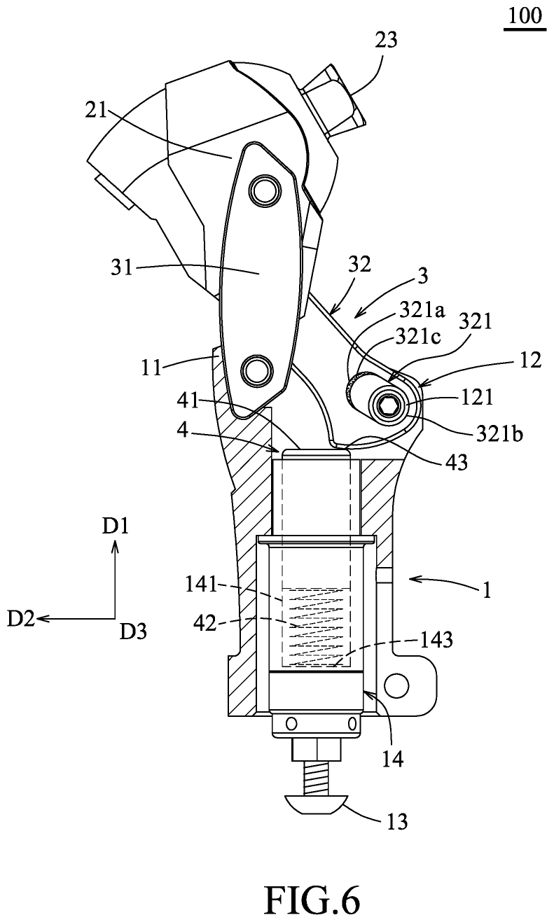

With reference to FIGS. 3 to 6, through pivoting of the linkage assembly 3, the thigh connecting part 2 is movable relative to the lower leg connecting part 1 between a stance position (see FIG. 3) and a knee bending position (see FIG. 5). In the stance position, the thigh socket is in an upright position relative to the shin tube, and, in this embodiment, the upper slot end section (321a) of the pivot linkage 321 is disposed slightly anterior of the lower slot end section (321b). In the knee bending position, an upper portion of the posterior link 32 is tilted forwards, a front section of the lower portion of the posterior link 32 is pressed downwardly the abutting member 41 of the biasing unit 4, and the thigh connecting part 2 is tilted slightly forwards. Thus, with a larger amount of tilt of the posterior link 32 relative to the thigh connecting part 2, the thigh connecting portion 23 is tilted rearwards so as to permit the thigh sleeve to bend relative to the shin tube. Also, in the knee bending position, the biasing member 42 of the biasing unit 4 has a biasing action to bring the abutting member 41 to bias upwardly the posterior link 32 at the front section of the lower portion, which resists further forward tilting of the upper portion of the posterior link 32 and urges the movement of the thigh connecting part 2 back toward the stance position.

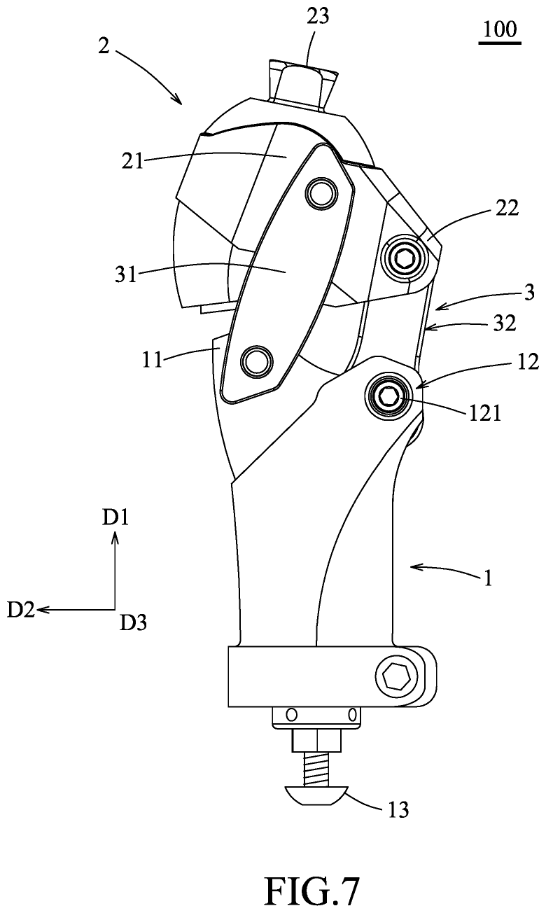

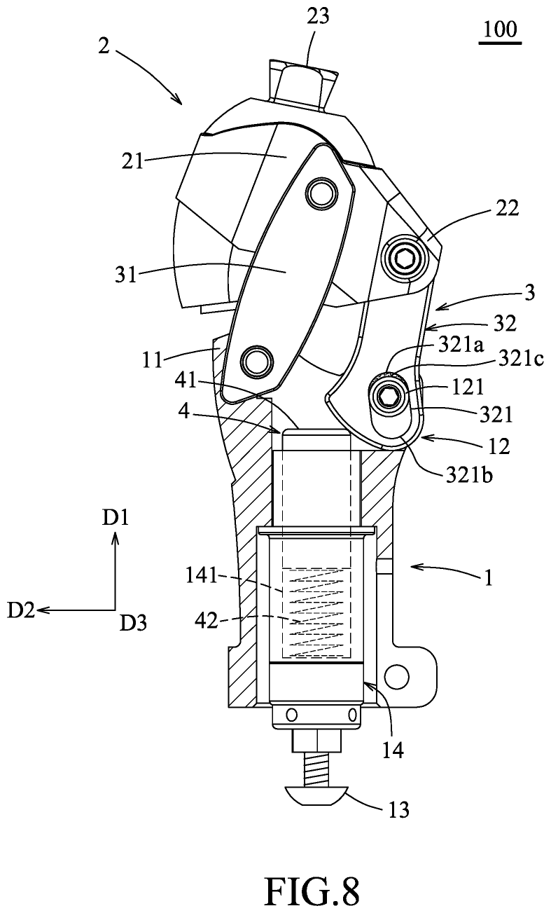

With reference to FIGS. 3, 4, 7 and 8, when an impact in the up-down direction (D1) is applied to the prosthetic knee joint 100 generated as a result of contact with the ground during walking, the posterior link 32 is moved downward relative to the lower leg connecting part 1 such that the upper slot end section (321a) of the pivot linkage 321 is moved downward to engage with the slidable pivot shaft 121, and the front section of the lower portion of the posterior link 32 is pressed downwardly against the abutting member 41 of the biasing unit 4, as shown in FIGS. 7 and 8. At this phase, a biasing action of the biasing member 42 is generated to bias the abutting member 41 upwardly so as to urge the posterior link 32 to move upward to bring the lower slot end section (321b) of the pivot linkage 321 to engage with the slidable pivot shaft 121. During this process, with the slidable engagement of the slidable pivot shaft 121 of the first posterior link portion 12 with the pivot linkage 321 of the posterior link 32, and the biasing unit 4 biasing the posterior link 32 upwardly and urging the pivot linkage 321 to bring the lower slot end section (321b) to engage with the slidable pivot shaft 121, a damping effect is provided to reduce the impact transmitted from the lower leg connecting part 1 when contacting with the ground. Additionally, with the cushion member (321c) disposed in the upper slot end section (321a), a further damping effect is provided when the slidable pivot shaft 121 is moved from the lower slot end section (321b) to the upper slot end section (321a). Moreover, during the movement of the thigh connecting part 2 between the stance position and the knee bending position, the damping effect is provided with the slidable engagement of the slidable pivot shaft 121 with the pivot linkage 321.

Furthermore, when the upper slot end section (321a) of the pivot linkage 321 is moved to engage with the slidable pivot shaft 121 during the pivoting of the linkage assembly 3, the angular position of the posterior link 32 relative to the anterior links 31 has been changed such that, after the thigh connecting part 2 is moved through the pivoting of the linkage assembly 3 relative to the lower leg connecting part 1 by a predetermined amount of movement, the pivoting movement of the linkage assembly 3 is resisted and the rearward tilting movement of the thigh connecting part 2 relative to the lower leg connecting part 1 is also resisted. The thigh socket is hence kept in a predetermined angular position relative to the shin tube. Thus, an excess inflexion of the thigh socket relative to the shin tube is prevented for increased safety. Also, the predetermined amount of movement and the predetermined position (i.e., a maximum knee angle) are adjustable as required. For example, the predetermined amount and the predetermined angular position can be adjusted by varying the structure of the linkage assembly 3 (e.g., length, initial angle, etc.) and/or by varying the pivot axes between the linkage assembly 3 and one of the lower leg connecting part 1 and the thigh connecting part 2.

As illustrated, with the slidable engagement of the slidable pivot shaft 121 of the first posterior link portion 12 with the pivot linkage 321 of the posterior link 32, and the biasing unit 4 biasing the posterior link 32 upwardly and urging the pivot linkage 321 to bring the lower slot end section (321b) to engage with the slidable pivot shaft 121, a damping effect is provided to reduce the impact when contacting with the ground. Also, an excess inflexion of the knee joint in the foot ground-contact phase is prevented for increased safety.

While the disclosure has been described in connection with what is considered the exemplary embodiment, it is understood that this disclosure is not limited to the disclosed embodiment but is intended to cover various arrangements included within the spirit and scope of the broadest interpretation so as to encompass all such modifications and equivalent arrangements.

What is claimed is:

1. A prosthetic knee joint comprising:
a lower leg connecting part having a first anterior link portion and a first posterior link portion, said first posterior link portion having a slidable pivot shaft which extends in a left-right direction;
a thigh connecting part disposed above said lower leg connecting part and having a second anterior link portion and a second posterior link portion;
a linkage assembly disposed between said lower leg connecting part and said thigh connecting part, said linkage assembly including at least one anterior link which is pivotably connected between said first anterior link portion and said second anterior link portion, and a posterior link which is pivotably connected between said first posterior link portion and said second posterior link portion, said posterior link having a slotted pivot linkage which extends therethrough in the left-right direction for pivotally connecting and engaging with said slidable pivot shaft, said pivot linkage being elongated in an up-down direction to have an upper slot end section and a lower slot end section, said slidable pivot shaft being slidably and rotatably engaged in said pivot linkage such that, through pivoting of said linkage assembly, said thigh connecting part is movable relative to said lower leg connecting part between a stance position and a knee bending position, and such that said slidable pivot shaft is moved from said lower slot end section to said upper slot end section with an impact transmitted from said lower leg connecting part to cause a downward movement of said posterior link relative to said lower leg connecting part; and
a biasing unit abutting against said posterior link to urge movement of said thigh connecting part toward the stance position, and to bias upwardly said posterior link so as to urge said lower slot end section of said pivot linkage to engage with said slidable pivot shaft.

2. The prosthetic knee joint as claimed in claim 1, wherein said biasing unit is attached to and abuts against a lower portion of said posterior link at a torque application point which is disposed anterior of said slidable pivot shaft.

3. The prosthetic knee joint as claimed in claim 2, wherein said lower leg connecting part has a central accommodation barrel which has an upper opened end and which extends in an up-down direction from said upper opened end to terminate at a bottom barrel wall, said central accommodation barrel defining an accommodation space therein, said biasing unit including an abutting member which is slidably received in said accommodation space and which projects partially from said upper opened end of said central accommodation barrel to abut against said posterior link, and a biasing member which is disposed in said accommodation space and interposed between said bottom barrel wall and said abutting member.

4. The prosthetic knee joint as claimed in claim 1, wherein said posterior link has a cushion member disposed in said upper slot end section for providing a damping effect when said slidable pivot shaft is moved from said lower slot end section to said upper slot end section.

5. The prosthetic knee joint as claimed in claim 1, wherein said upper slot end section is disposed anterior of said lower slot end section when said thigh connecting part is in the stance position.

6. The prosthetic knee joint as claimed in claim 1, wherein said slidable pivot shaft is moved from said lower slot end section to said upper slot end section by a distance ranging from 5 mm to 7 mm.

7. The prosthetic knee joint as claimed in claim 6, wherein the distance is 6 mm.
